(19) United States
(12) Patent Application Publication
(10) Pub. No.: US 2014/0288502 A1
(43) Pub. Date: Sep. 25, 2014

(54) INTRAVASCULAR CATHETER

(71) Applicant: Mauro Massafumi TANIWAKI, Sao Paulo (BR)

(72) Inventor: Mauro Massafumi TANIWAKI, Sao Paulo (BR)

(21) Appl. No.: 14/222,871

(22) Filed: Mar. 24, 2014

(30) Foreign Application Priority Data
Mar. 25, 2013 (BR) 102013006802

(51) Int. Cl. A61M 25/00; A61L 29/04; A61L 29/14

(52) U.S. Cl. CPC A61M 25/0023; A61L 29/14; A61L 29/049; A61M 2025/0024; A61M 25/0606
USPC 604/164.13

(57) ABSTRACT

This abstract relates to an angiographic, central venous, balloon expansion, peripheral over the needle intravascular catheter, belonging to the field of medical devices comprising the catheter itself, which is inserted into the body, obtained from a suitable material to have a flow proportional to the temperature.

### BACKGROUND OF THE DISCLOSURE

This specification relates to a patent of invention for an angiographic intravascular, central venous, balloon expansion, peripheral over the needle catheter or other belonging to the field of medical devices, which has been improved to provide a flow proportional to temperature.

Intravascular catheters are already known in the art by essentially comprising: a flexible cannula (main lumen) made of silicone, polyethylene, polyurethane, plastic or the like, having a distal end that is introduced into the body, and in opposition thereto, one or multiple lumens; said cannula being designed to be either partially or wholly inserted or implanted in the cardiovascular system of the patient for diagnostic and/or therapeutic purposes. Typically, said cannula, which is part of the catheter, has a rated flow for application or administration of the solution under suitable conditions for proper incorporation by the body.

However, there are medical events, such as bleeding, decreased blood pressure, tissue perfusion or others, where it is desirable a more rapid delivery rate of drugs, solutions, blood and derivatives. Smaller diameter and lower flow catheters are unable to suitably meet such requirements, as their flow is not always compatible with the urgency of the case.

Thus, the object of this patent of invention is to provide a catheter designed to overcome the above-mentioned drawback.

Another object of the invention is to provide a catheter, which, besides overcoming such drawback is not for this very same reason constructively more complex in such way to become uninteresting over typical catheters.

Another object of the invention is to provide a catheter at a suitable cost.

### SUMMARY OF THE INVENTION

Therefore, in view of the above-mentioned drawback, and in order to overcome it while achieving the other related objects of this invention, the improved intravascular catheter object of this patent of invention has been designed to be made from the same type of material as that used for common catheters, i.e. silicone, polyethylene, polyurethane, plastic or others, having a selected coefficient of expansion so that at room temperature the catheter gauge (diameter) and flow are lower, and at the temperature inside the human body, the catheter expands and the gauge and flow increase accordingly.

Initially, at room temperature or lower, the diameter and flexibility of the catheter with such a construction are smaller, what favors inserting and maneuvering the catheter inside the body; otherwise, after introduction and exposure to the temperature inside the body, the catheter expands and achieves a larger diameter and flow, what favors a more rapid delivery of the drug into the patient, according to the main object of the invention. This catheter system is particularly convenient for medical procedures where a more rapid delivery rate is desirable, such as in cases of bleeding, decreased blood pressure, tissue perfusion or other events during which time is the key for the maintenance of the good conditions of the patient.

Besides the advantages provided by this catheter, and in order to accomplish the other objects of the invention, its construction and manufacturing have substantially the same levels of complexity and costs as typical catheters.

### BRIEF DESCRIPTION OF THE DRAWINGS

The attached drawings refer to the improvement in intravascular catheter object of the present invention, in which:

FIG. 1 shows the central intravascular catheter assembly at rest, for informational and non-restrictive purposes;

FIG. 2 shows a section of the previous figure, in which the section of the cannula that is inserted into the body is seen forming the catheter assembly, which is shown at rest at room temperature and with a smaller diameter;

FIG. 3 shows a peripheral intravascular catheter and the indication of its application inside the body; and

FIG. 4 shows a sectional view of the catheter shown in the previous figure, in which its section is seen and the indication of its expansion when subject to the temperature existing inside the body.

### DETAILED DESCRIPTION OF THE DRAWINGS

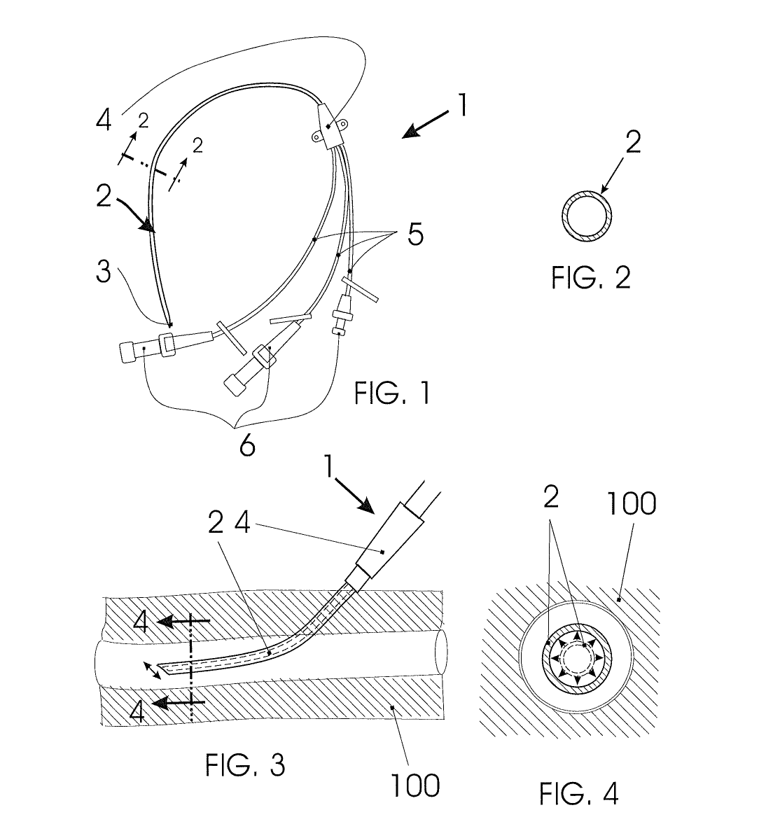

As shown in the above-mentioned figures and provided in the invention, the intravascular catheter 1 object of this patent of invention is of the type used for IV administration of solutions, and it may be central venous (FIG. 1) or peripheral over the needle (FIG. 3) or angiographic (not shown) or balloon expansion (not shown) catheter or other and it is essentially comprised by: the catheter itself 2 comprising a flexible cannula (main lumen) made from a material such as silicone, polyethylene, polyurethane, plastic or the like, which is inserted into the body, having a free distal end 3 and an opposite end which may be mounted in a junction 4; and one or more flexible guidewires 5 (lumens) connected to proximal end(s) 6 featuring a hypodermal needle type connection with which the equipment (not shown), such as syringe(s) or others containing and delivering injectable solution(s) are coupled.

This improvement comprises manufacturing the catheter itself 2 to be introduced into the body such that it has a flow directly proportional to the temperature.

For such purpose, the catheter 2 is made of a material such as: silicone, polyethylene, polyurethane, plastic or the like with a selected expansion index so that at room temperature, the gauge (diameter) and flow of the catheter are smaller, while when exposed to the temperature inside the body it expands and achieves a larger gauge and flow to provide greater flow for faster injection of the solution.

Describing the details of the invention, the catheter 2 has an appropriate expansion index so that at room temperature it has the usual diameter for intravascular catheters and achieves a maximum larger diameter at the patient's body temperature.

The materials from which the components of the catheter assembly are made are nontoxic, biocompatible and suitable for medical use.

All other components, such as protective capsule, housing and other typical components are part of the catheter assembly.

The catheter is applied by a healthcare provider according to usual directions for this type of device. Thus, during application, while still at room temperature, the catheter itself 2 to be introduced into the patient's body has a smaller diameter (FIGS. 1, 2), which facilitates its application and maneuvering inside the body. At the end of the application, when the catheter 2 is already inside the body 100 (FIG. 3) and subjected to the patient's body temperature, the catheter 2 expands, thus increasing its diameter and flow (FIG. 4), and thereby providing faster delivery, according to the object hereof

Within the basic construction described above, the intravascular catheter object of the present application may have its materials, dimensions, construction details and/or functional configuration modified or changed without departing from the scope of protection sought.

Therefore, as already mentioned herein, the intravascular catheter may be: an angiographic, central venous, balloon expansion, peripheral over the needle catheter, according to standard NBR ISO 10555:2003 for “Single-use intravascular catheter”, the configuration of the parts of such catheter being suitable for this type of catheter and further incorporating the object of this patent.

What is claimed:

1. An intravascular catheter, comprising a flexible cannula made of silicone, polyethylene, polyurethane, plastic, which is introduced into the body, having a free distal end and an opposite end which may incorporate a junction and one or more flexible guidewires connected to proximal end(s) with a hypodermal needle connection, where equipment containing and delivering injectable solution(s) are coupled, characterized in that the catheter which is inserted into the body has a flow directly proportional to the temperature.

2. The intravascular catheter, as claimed in claim 1, characterized in that the catheter is obtained from: silicone, polyethylene, polyurethane or plastic with an expansion index selected to have a lower gauge and flow at room temperature and expand at the temperature inside the human body, thereby achieving larger gauge and flow.

3. The intravascular catheter, as claimed in claim 1, characterized in that the catheter has the suitable expansion index to achieve, at room temperature, a first diameter for intravascular catheters, and to expand and achieve a second diameter at the patient body's temperature, wherein the second diameter is larger than the first diameter.
